(19) United States
(12) Patent Application Publication
(10) Pub. No.: US 2023/0346291 A1
(43) Pub. Date: Nov. 2, 2023

(54) RRI Measurement Device, RRI Measurement Method and RRI Measurement Program

(71) Applicant: NIPPON TELEGRAPH AND TELEPHONE CORPORATION, Tokyo (JP)

(72) Inventors: Yuki Hashimoto, Tokyo (JP); Kei Kuwabara, Tokyo (JP); Nobuaki Matsuura, Tokyo (JP)

(21) Appl. No.: 18/247,848

(22) PCT Filed: Oct. 27, 2020
(86) PCT No.: PCT/JP2020/040202

§ 371 Date: Apr. 4, 2023

(51) Int. Cl. A61B 5/352; A61B 5/00; A61B 5/366

(52) U.S. Cl. CPC A61B 5/352; A61B 5/7225; A61B 5/366

(57) ABSTRACT

An RRI measurement device includes: a feature point detection unit configured to detect a feature point derived from an RS wave from a sampling data sequence of an electrocardiogram signal of a subject; a time estimation unit configured to search for two sampling points where a product of adjacent pieces of sampling data is zero or less in sampling data near the feature point and to estimate a time at which the electrocardiogram signal becomes zero using the two sampling points; and an RRI calculation unit configured to calculate an RRI on the basis of time-series data of the time estimated by the time estimation unit.

### CROSS-REFERENCE TO RELATED APPLICATIONS

This application is a national phase entry of PCT Application No. PCT/JP2020/040202, filed on Oct. 27, 2020, which application is hereby incorporated herein by reference.

### TECHNICAL FIELD

The present invention relates to an RRI measurement device, an RRI measurement method, and an RRI measurement program that detect an RRI that is an interval between two adjacent R waves from an electrocardiogram signal.

### BACKGROUND

An R-R interval (RRI) in an electrocardiogram (ECG) is used for analysis of heart rate variability, diagnosis of arrhythmia, detection of heart disease and disorder of the automatic nerve system, and the like. With recent development of technology, real-time ECG acquisition and RRI detection have become possible by using a wearable device (see Non Patent Literature 1).

For example, there has been proposed an application that acquires time-series data of an ECG signal while clothes are worn by using a wearable device attached to the clothes, calculates a heart rate and an RRI on the basis of the ECG signal, and wirelessly transmits obtained data group to an external terminal such as a smartphone (see Patent Literature 1).

In general, accuracy of detecting an RRI from an ECG signal depends on a sampling rate of the ECG signal, and the analysis of the heart rate variability requires the sampling rate of 2 msec. or more, that is, 500 samples/sec. or more.

Meanwhile, in a case where signal acquisition is performed at the above high sampling rate in the wearable device, a calculation throughput increases and power consumption of the device increases, which are problematic.

### CITATION LIST

### Patent Literature

Patent Literature 1: JP 2016-24495 A

### Non Patent Literature

Non Patent Literature 1: M. Elgandi et al., “Revisiting QRS Detection Methodologies for Portable, Wearable, Battery-Operated, and Wireless ECG Systems”, PLOS ONE, Vol. 9, No. 1, e84018, 2014, DOI: 10.1371/journal. pone. 0084018

### SUMMARY

### Technical Problem

Embodiments of the present invention have been made to solve the above problems, and an object of embodiments of the present invention is to provide an RRI measurement device, an RRI measurement method, and an RRI measurement program capable of accurately measuring an RRI of a subject on the basis of sampling data of an electrocardiogram signal acquired at a low sampling rate.

### Solution to Problem

An RRI measurement device according to embodiments of the present invention includes: a detection unit configured to detect a feature point derived from an RS wave from a sampling data sequence of an electrocardiogram signal of a subject; an estimation unit configured to search for two sampling points where a product of adjacent pieces of sampling data is zero or less in sampling data near the feature point and to estimate a time at which the electrocardiogram signal becomes zero using the two sampling points; and a calculation unit configured to calculate an RRI on the basis of time-series data of the time estimated by the estimation unit.

Further, an RRI measurement device according to embodiments of the present invention includes: a detection unit configured to detect a feature point derived from an RS wave from a sampling data sequence of an electrocardiogram signal of a subject; an estimation unit configured to search for two time difference values where a product of adjacent time difference values is zero or less among time difference values of sampling data near the feature point and to estimate a time at which the electrocardiogram signal becomes zero using the two time difference values; and a calculation unit configured to calculate an RRI on the basis of time-series data of the time estimated by the estimation unit.

An RRI measurement method according to embodiments of the present invention includes: a first step of detecting a feature point derived from an RS wave from a sampling data sequence of an electrocardiogram signal of a subject; a second step of searching for two sampling points where a product of adjacent pieces of sampling data is zero or less in sampling data near the feature point and estimating a time at which the electrocardiogram signal becomes zero using the two sampling points; and a third step of calculating an RRI on the basis of time-series data of the time estimated in the second step.

Further, an RRI measurement method according to embodiments of the present invention includes: a first step of detecting a feature point derived from an RS wave from a sampling data sequence of an electrocardiogram signal of a subject; a second step of searching for two time difference values where a product of adjacent time difference values is zero or less among time difference values of sampling data near the feature point and estimating a time at which the electrocardiogram signal becomes zero using the two time difference values; and a third step of calculating an RRI on the basis of time-series data of the time estimated in the second step.

Further, an RRI measurement program of embodiments of the present invention causes a computer to execute each of the above steps.

### ADVANTAGEOUS EFFECTS OF EMBODIMENTS OF INVENTION

According to embodiments of the present invention, providing a detection unit, an estimation unit, and a calculation unit makes it possible to accurately measure an RRI of a subject on the basis of sampling data of an electrocardiogram signal acquired at a low sampling rate. In embodiments of the present invention, it is unnecessary to acquire an electrocardiogram signal at a high sampling rate, and thus it is possible to reduce power consumption of an RRI measurement device.

### BRIEF DESCRIPTION OF THE DRAWINGS

FIG. 1 is a block diagram showing a configuration of an RRI measurement device according to a first embodiment of the present invention.

FIG. 2 is a functional block diagram of an MCU of the RRI measurement device according to the first embodiment of the present invention.

FIG. 3 is a flowchart showing an operation of the MCU of the RRI measurement device according to the first embodiment of the present invention.

FIG. 4 is a waveform diagram showing an example of an ECG signal.

FIGS. 5A and 5B are explanatory diagrams showing processing of a time estimation unit of the RRI measurement device according to the first embodiment of the present invention.

FIG. 6 is a functional block diagram of an MCU of an RRI measurement device according to a second embodiment of the present invention.

FIG. 7 is a flowchart showing an operation of the MCU of the RRI measurement device according to the second embodiment of the present invention.

FIGS. 8A to 8C are explanatory diagrams showing processing of a time estimation unit of the RRI measurement device according to the second embodiment of the present invention.

FIG. 9 is a block diagram showing a configuration example of a computer that implements the RRI measurement devices according to the first and second embodiments of the present invention.

### DETAILED DESCRIPTION OF ILLUSTRATIVE EMBODIMENTS

### First Embodiment

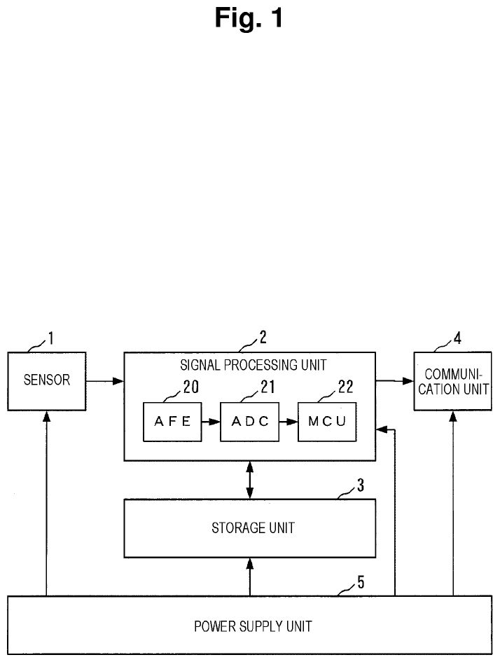

Hereinafter, an embodiment of the present invention will be described with reference to the drawings. FIG. 1 is a block diagram showing a configuration of an RRI measurement device according to a first embodiment of the present invention. The RRI measurement device includes a sensor 1, a signal processing unit 2, a storage unit 3, a communication unit 4, and a power supply unit 5.

The signal processing unit 2 includes an analog front end (AFE) 20, an analog digital converter (ADC) 21, and a micro control unit (MCU) 22.

The sensor 1 detects an ECG signal of a subject. The AFE 20 amplifies a weak ECG signal detected by the sensor 1.

The ADC 21 converts the ECG signal amplified by the AFE 20 into digital data at a predetermined sampling rate. Examples of the sampling rate include 125 samples/sec. and 250 samples/sec.

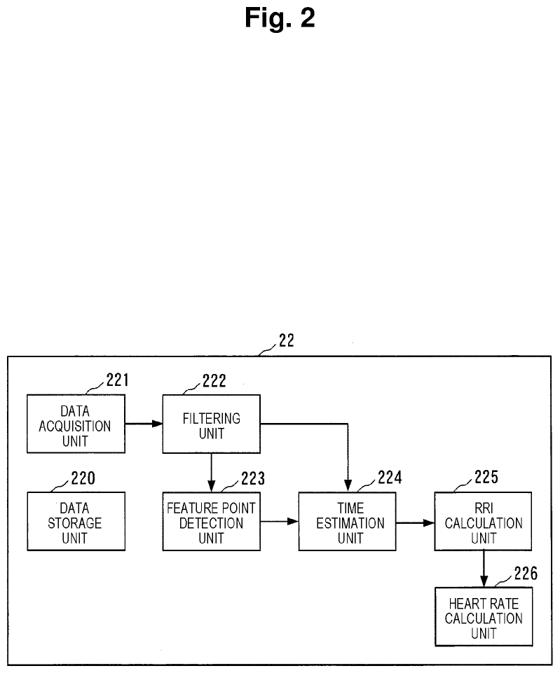

The MCU 22 is a circuit that performs signal processing for calculating an RRI. FIG. 2 is a functional block diagram of the MCU 22. The MCU 22 functions as a data storage unit 220, a data acquisition unit 221, a filtering unit 222, a feature point detection unit 223, a time estimation unit 224, an RRI calculation unit 225, and a heart rate calculation unit 226.

The storage unit 3 stores a program of the MCU 22, sampling data of an ECG signal output from the ADC 21, and data calculated by the MCU 22.

The data storage unit 220 of the MCU 22 adds sampling time information to each piece of sampling data of ECG signals output from the ADC 21 and stores the sampling data in the storage unit 3.

The communication unit 4 includes a circuit that wirelessly transmits the sampling data of the ECG signal and the data calculated by the MCU 22 to an external device (not illustrated) such as a smartphone.

The power supply unit 5 is a circuit that supplies power to the entire RRI measurement device.

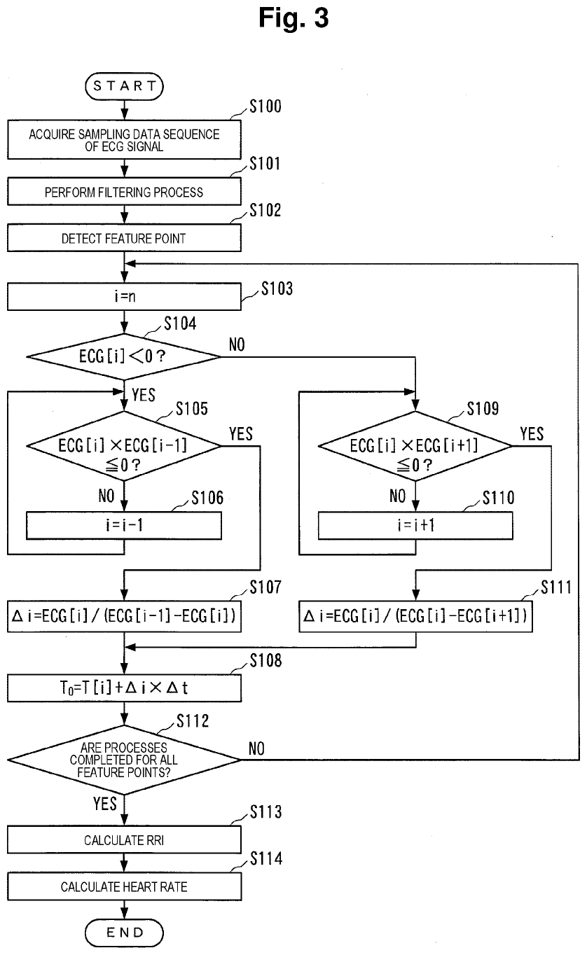

FIG. 3 is a flowchart showing an operation of the MCU 22. The data acquisition unit 221 acquires a sampling data sequence of an ECG signal stored in the storage unit 3 (step S100 in FIG. 3).

The filtering unit 222 performs a filtering process by using an anti-aliasing filter or band-pass filter on the sampling data sequence acquired by the data acquisition unit 221 (step S101 in FIG. 3).

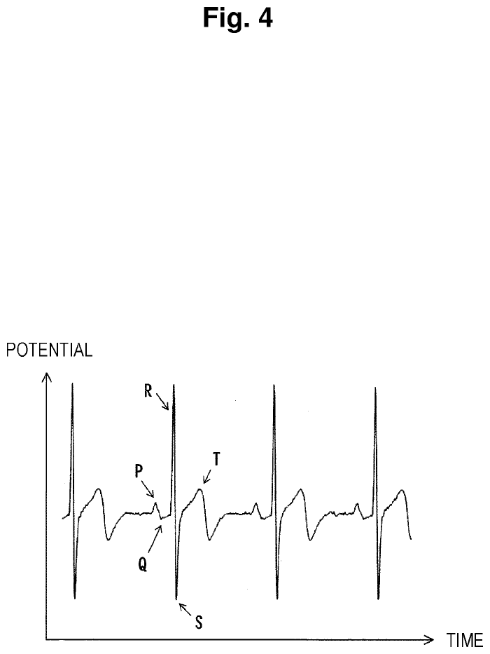

The feature point detection unit 223 detects a feature point derived from an RS wave from the sampling data sequence subjected to the filtering process (step S102 in FIG. 3). As shown in FIG. 4, the ECG signal has a continuous heartbeat waveform, and one heartbeat waveform includes components such as a P wave, Q wave, R wave, S wave, and T wave each of which reflects activity of atria or ventricles.

In the present embodiment, a value (potential) of the sampling data of the ECG signal is denoted by ECG[n], and a sampling time is denoted by T[n]. The letter “n” is a number given to data of one sampling. It is needless to say that the sampling time is later as the number n is larger.

As a method of detecting the feature point, a method using a threshold is simple. The feature point detection unit 223 only needs to detect a section in which the sampling data ECG[n] exceeds a predetermined positive threshold for detecting the R wave as a section of the R wave and detect any one representative point of the sampling data ECG[n] in the section as the feature point. For example, a peak point having the highest potential may be used as the feature point, or a first sampling point exceeding the threshold may be used as the feature point. The feature point detection unit 223 detects such a feature point in each section of the R wave.

The feature point detection unit 223 may detect the feature point by another method. Another method is, for example, a method of using a time difference value (first derivative) of an ECG signal.

The feature point detection unit 223 calculates a time difference value dECG[n] of the sampling data ECG[n] as in the following expression by using the sampling data ECG[n] and data ECG[n−1] one sampling therebefore.

dECG[n] =ECG[n]−ECG[n−1] (1)

The feature point detection unit 223 calculates the time difference value dECG[n] at each sampling time (each piece of the sampling data). For the time difference value dECG[n], a peak caused by a steep change from the R wave to the S wave appears as a negative value.

The feature point detection unit 223 only needs to detect a section in which the time difference value dECG[n] is lower than a predetermined negative threshold for detecting the RS wave as a section from the R wave to the S wave and detect any one representative point of the time difference values dECG[n] in the section as the feature point. For example, a peak point having the lowest potential may be used as the feature point, or a first time difference value dECG[n] that becomes lower than the threshold may be used as the feature point. When detecting a negative peak point of the time difference value dECG[n], the feature point detection unit 223 may set a positive peak point immediately before the negative peak point as a peak point of the R wave and detect the peak point of the R wave as the feature point. The feature point detection unit 223 detects the feature point in each section from the R wave to the S wave.

The detection method described above is disclosed in Non Patent Literature 1. Further, the method of detecting the feature point is not limited to the method of the present embodiment, and the feature point may be detected by a method other than the method of the present embodiment.

Next, the time estimation unit 224 estimates a time at which the ECG signal becomes zero on the basis of the feature point detected by the feature point detection unit 223 and sampling data near the feature point.

Specifically, the time estimation unit 224 initializes a number i for specifying a base point of time calculation to a number n of the sampling data ECG[n] detected as the feature point by the feature point detection unit 223 and sets the sampling data ECG[n] as the base point of the time calculation (step S103 in FIG. 3).

Then, the time estimation unit 224 determines whether or not sampling data ECG[i] of the base point is smaller than zero (step S104 in FIG. 3). When the sampling data ECG[i] is smaller than zero (YES in step S104), the time estimation unit 224 determines whether or not a product of the sampling data ECG[i] and data ECG[i−1] one sampling therebefore is zero or less (step S105 in FIG. 3). When the product is not zero or less (NO in step S105), the time estimation unit 224 decreases the number i for specifying the base point by 1 (step S106 in FIG. 3), and the processing returns to step S105.

In this way, a position of the base point is shifted forward by one sampling until the product ECG[i]×ECG[i−1] of the sampling data ECG[i] of the base point and the sampling data ECG[i−1] one sampling therebefore becomes zero or less. When the product is zero or less (YES in step S105), the time estimation unit 224 calculates a ratio Δi obtained by dividing a difference between a time T[i] of the base point and a time at which the ECG signal becomes zero by a sampling period Δt from the following expression (step S107 in FIG. 3).

Δi=ECG[i]/(ECG[i−1]−ECG[i]) (2)

Then, the time estimation unit 224 sets a result of adding a value obtained by multiplying the ratio Δi by the sampling period Δt to the time T[i] of the base point as a time T₀ at which the ECG signal becomes zero (step S108 in FIG. 3).

T₀=T[i]+Δi×Δt (3)

Meanwhile, when determining in step S104 that the sampling data ECG[i] of the base point is zero or more, the time estimation unit 224 determines whether or not a product of the sampling data ECG[i] and data ECG[i+1] one sampling thereafter is zero or less (step S109 in FIG. 3). When the product is not zero or less (NO in step S109), the time estimation unit 224 increases the number i for specifying the base point by 1 (step S108 in FIG. 3), and the processing returns to step S109.

In this way, the position of the base point is shifted backward by one sampling until the product ECG[i]×ECG[i+1] of the sampling data ECG[i] of the base point and the sampling data ECG[i+1] one sampling thereafter becomes zero or less. When the product is zero or less (YES in step S109), the time estimation unit 224 calculates the ratio Δi obtained by dividing the difference between the time T[i] of the base point and the time at which the ECG signal becomes zero by the sampling period Δt from the following expression (step S111 in FIG. 3).

Δi=ECG[i]/(ECG[i]−ECG[i+1]) (4)

The process in step S108 is as described above. The time estimation unit 224 performs the processes in steps S103 to S111 for each feature point until the processes are completed for all the feature points detected by the feature point detection unit 223 (YES in step S112 in FIG. 3). Time-series data of the time T₀ is stored in the storage unit 3.

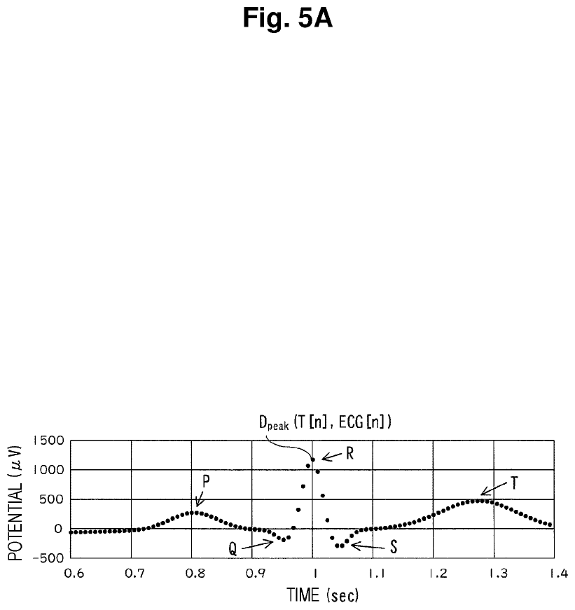

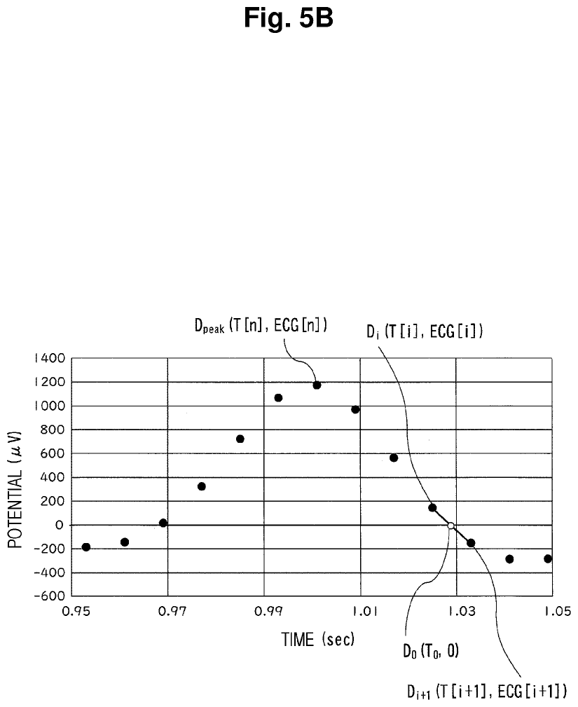

FIGS. 5A and 5B are explanatory diagrams showing processing of the time estimation unit 224. FIG. 5A shows an example of the sampling data sequence of the ECG signal, and FIG. 5B is an enlarged view of a section from 0.95 seconds to 1.05 seconds in FIG. 5A.

For example, in a case where a peak point Dpeak of the R wave is detected as the feature point, the time estimation unit 224 searches for two sampling points where a product of adjacent pieces of the sampling data is zero or less in the sampling data near the feature point and calculates the time T₀ at which the ECG signal becomes zero using the two sampling points. In the example of FIG. 5B, a product of the sampling data ECG[i] of a base point Dᵢ and the data ECG[i+1] of a point Dᵢ₊₁, one sampling thereafter is zero or less, and thus the time T₀ at which the ECG signal becomes zero is calculated by the processes in steps S111 and S108.

Next, the RRI calculation unit 225 calculates a time interval between two adjacent times T₀ as an RRI on the basis of the time-series data of the times T₀ stored in the storage unit 3 (step S113 in FIG. 3). The RRI calculation unit 225 calculates the RRI at each time T₀. Time-series data of the calculated RRIs is stored in the storage unit 3. The RRI calculation unit 225 may calculate not only the RRIs but also an average value of the RRIs.

The heart rate calculation unit 226 calculates an instantaneous heart rate X (beats/min.) for each RRI on the basis of the RRIs calculated by the RRI calculation unit 225 (step S114 in FIG. 3).

X=60000/RRI (5)

Time-series data of the calculated instantaneous heart rate X is stored in the storage unit 3. The heart rate calculation unit 226 may calculate not only the instantaneous heart rate X but also an average value of the instantaneous heart rate X.

The communication unit 4 wirelessly transmits the sampling data sequence of the ECG signal, the time-series data of the RRIs and the average value of the RRIs calculated by the RRI calculation unit 225, and the time-series data of the instantaneous heart rate X and the average value of the instantaneous heart rate X calculated by the heart rate calculation unit 226 to the external device such as a smartphone.

Therefore, in the present embodiment, it is possible to accurately measure the RRIs and the heart rate of the subject on the basis of the sampling data of the ECG signal acquired at a low sampling rate such as 125 samples/sec. or 250 samples/sec. In the present embodiment, it is unnecessary to acquire the ECG signal at a sampling rate higher than 500 samples/sec., and thus it is possible to reduce power consumption of the RRI measurement device.

### Second Embodiment

Next, a second embodiment of the present invention will be described. Also in the present embodiment, a configuration of the entire RRI measurement device is similar to that of the first embodiment. Thus, the description will be made by using the reference signs of FIG. 1.

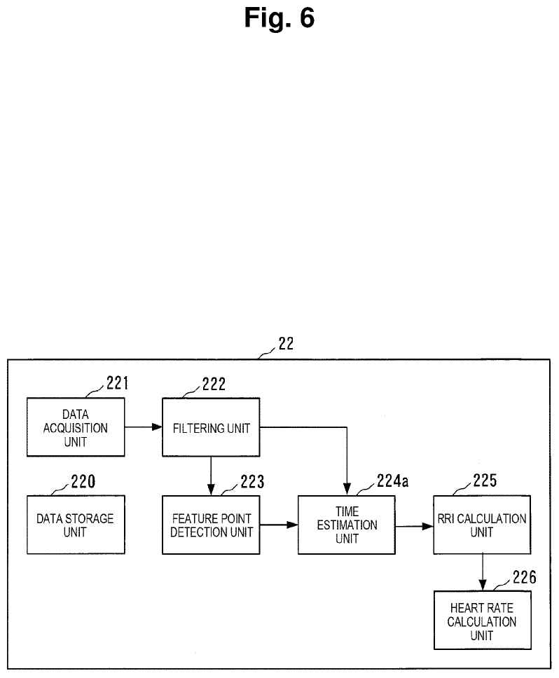

FIG. 6 is a functional block diagram of the MCU 22 in the present embodiment. The MCU 22 in the present embodiment functions as the data storage unit 220, the data acquisition unit 221, the filtering unit 222, the feature point detection unit 223, a time estimation unit 224a, the RRI calculation unit 225, and the heart rate calculation unit 226.

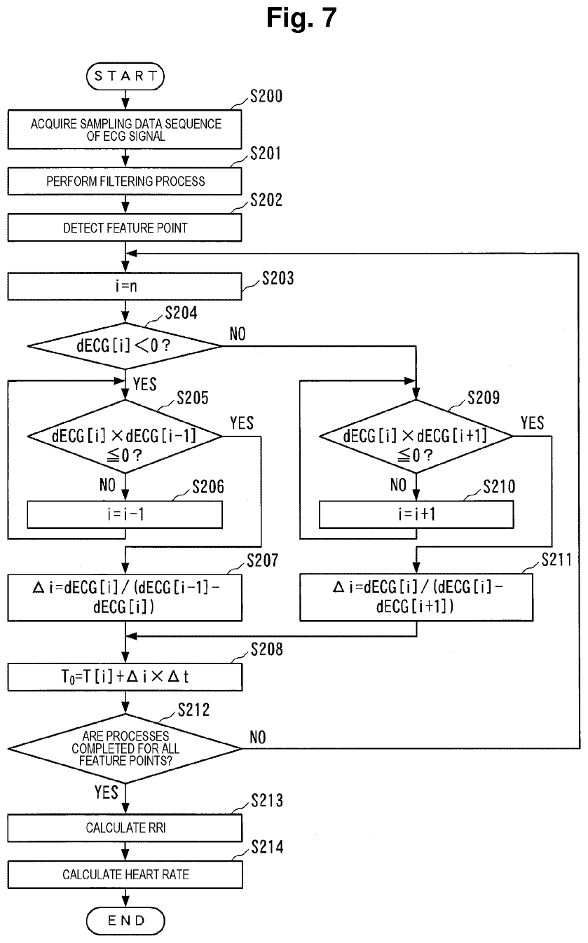

FIG. 7 is a flowchart showing an operation of the MCU 22. The data acquisition unit 221 acquires a sampling data sequence of an ECG signal stored in the storage unit 3 (step S200 in FIG. 7).

The filtering unit 222 performs a filtering process by using an anti-aliasing filter or band-pass filter on the sampling data sequence acquired by the data acquisition unit 221 (step S201 in FIG. 7).

As in the first embodiment, the feature point detection unit 223 detects a feature point derived from an RS wave from the sampling data sequence subjected to the filtering process (step S202 in FIG. 7).

The time estimation unit 224a estimates a time at which the ECG signal becomes zero on the basis of a time difference value of sampling data of the feature point detected by the feature point detection unit 223 and a time difference value of sampling data near the feature point.

Specifically, the time estimation unit 224a initializes a number i for specifying a base point of time calculation to a number n of the sampling data ECG[n] detected as the feature point by the feature point detection unit 223 and sets a time difference value dECG[i] of the sampling data ECG[i] as the base point of the time calculation (step S203 in FIG. 7).

dECG[i]=ECG[i]−ECG[i−1] (6)

Then, the time estimation unit 224a determines whether or not the time difference value dECG[i] of the sampling data ECG[i] of the base point is smaller than zero (step S204 in FIG. 7). When the time difference value dECG[i] is smaller than zero (YES in step S204), the time estimation unit 224a determines whether or not a product of the time difference value dECG[i] and a time difference value dECG[i−1] one sampling therebefore is zero or less (step S205 in FIG. 7). When the product is not zero or less (NO in step S205), the time estimation unit 224a decreases the number i for specifying the base point by 1 (step S206 in FIG. 7), and the processing returns to step S205.

In this way, a position of the base point is shifted forward by one sampling until the product dECG[i]×dECG[i−1] of the time difference value dECG[i] and the time difference value dECG[i−1] one sampling therebefore becomes zero or less. When the product is zero or less (YES in step S205), the time estimation unit 224a calculates the ratio Δi obtained by dividing the difference between the time T[i] of the base point and the time at which the ECG signal becomes zero by the sampling period Δt from the following expression (step S207 in FIG. 7).

Δi=dECG[i]/(dECG[i−1]−dECG[i]) (7)

Then, the time estimation unit 224a sets a result of adding a value obtained by multiplying the ratio Δi by the sampling period Δt to the time T[i] of the base point as a time T₀ at which the ECG signal becomes zero (step S208 in FIG. 7). A calculation expression of the time T₀ is the same as Expression (3).

Meanwhile, when determining in step S204 that the time difference value dECG[i] of the sampling data dECG[i] of the base point is zero or more, the time estimation unit 224a determines whether or not the product of the time difference value dECG[i] and the time difference value dECG[i+1] one sampling thereafter is zero or less (step S209 in FIG. 7). When the product is not zero or less (NO in step S209), the time estimation unit 224a increases the number i for specifying the base point by 1 (step S210 in FIG. 7), and the processing returns to step S209.

In this way, the position of the base point is shifted backward by one sampling until the product dECG[i]×dECG[i+1] of the time difference value dECG[i] and the time difference value dECG[i+1] one sampling thereafter becomes zero or less. When the product is zero or less (YES in step S209), the time estimation unit 224a calculates the ratio Δi obtained by dividing the difference between the time T[i] of the base point and the time at which the ECG signal becomes zero by the sampling period At from the following expression (step S211 in FIG. 7).

Δi=dECG[i]/(dECG[i]−dECG[i+1]) (8)

The process in step S208 is as described above. The time estimation unit 224a performs the processes in steps S203 to S211 for each feature point until the processes are completed for all the feature points detected by the feature point detection unit 223 (YES in step S212 in FIG. 7). Time-series data of the time T₀ is stored in the storage unit 3.

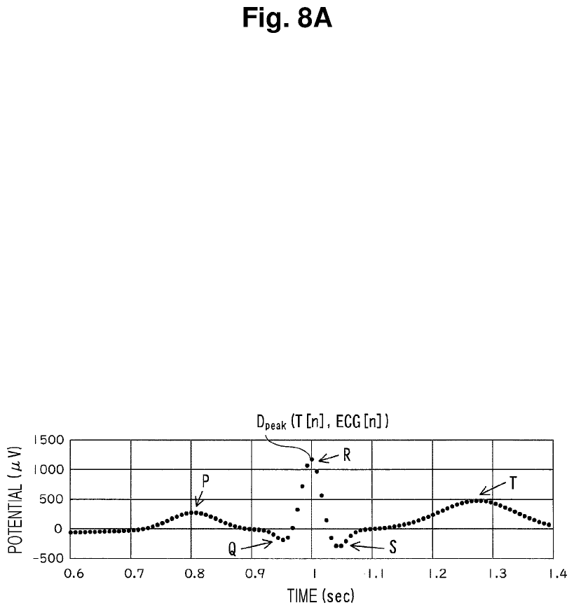

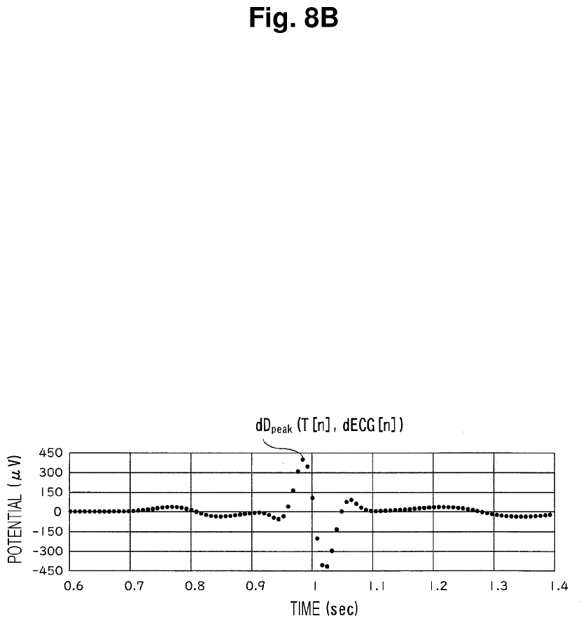

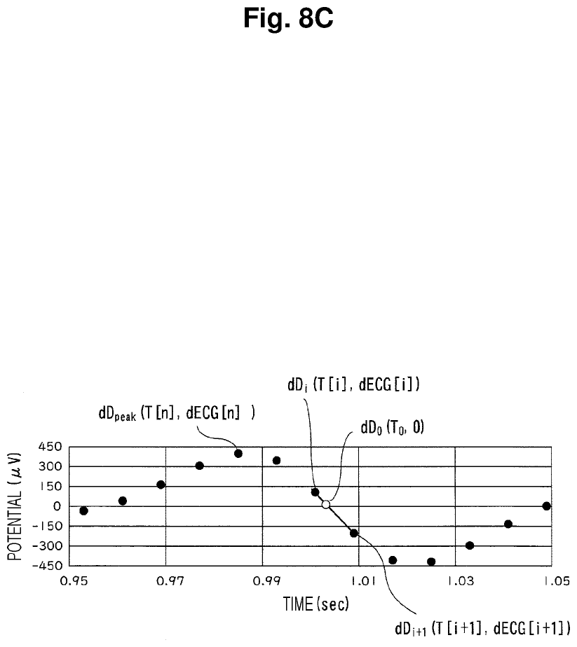

FIGS. 8A to 8C are explanatory diagrams showing processing of the time estimation unit 224a. FIG. 8A shows an example of the sampling data sequence of the ECG signal, FIG. 8B shows a time difference value calculated from the sampling data sequence of FIG. A, and FIG. 8C is an enlarged view of a section from 0.95 seconds to 1.05 seconds in FIG. 8B.

For example, when the peak point Dpeak of the R wave is detected as the feature point, dDpeak corresponds to feature points in FIGS. 8B and 8C. The time estimation unit 224a searches for two time difference values where a product of adjacent time difference values is zero or less among time difference values of sampling data near the feature point and calculates the time T₀ at which the ECG signal becomes zero using the two time difference values. In the example of FIG. 8C, a product of the time difference value dECG[i] of a base point dDi and the time difference value dECG[i+1] of a point dDi+1 one sampling thereafter is zero or less, and thus the time T₀ at which the ECG signal becomes zero is calculated by the processes in steps S211 and S208.

As in the first embodiment, the RRI calculation unit 225 calculates a time interval between two adjacent times T₀ as an RRI from the time-series data of the times T₀ stored in the storage unit 3 (step S213 in FIG. 7).

A process of the heart rate calculation unit 226 (step S214 in FIG. 7) is the same as that in the first embodiment.

The communication unit 4 wirelessly transmits the sampling data sequence of the ECG signal, the time-series data of the RRIs and the average value of the RRIs calculated by the RRI calculation unit 225, and the time-series data of the instantaneous heart rate X and the average value of the instantaneous heart rate X calculated by the heart rate calculation unit 226 to the external device such as a smartphone.

Therefore, effects similar to those of the first embodiment can be obtained in the present embodiment. In the first and second embodiments, the feature point is detected after the sampling data sequence of the ECG signal is subjected to the filtering process, but the filtering unit 222 is not an essential component in the present invention.

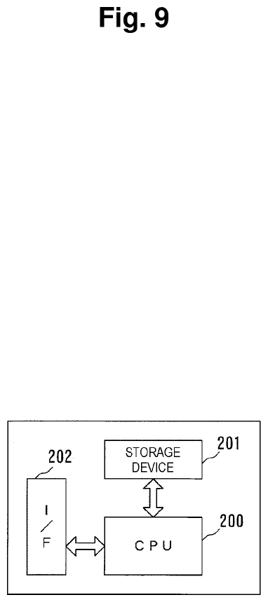

The storage unit 3 and the MCU 22 described in the first and second embodiments can be implemented by a computer including a central processing unit (CPU), a storage device, and an interface and a program for controlling those hardware resources. A configuration example of the computer is illustrated in FIG. 9. The computer includes a CPU 200, a storage device 201, and an interface device (hereinafter, abbreviated as the I/F) 202. The I/F 202 is connected to the ADC 21, the communication unit 4, and the like. In such a computer, an RRI measurement program for implementing the RRI measurement method of the present invention is stored in the storage device 201. The CPU 200 executes the processes described in the first and second embodiments according to the program stored in the storage device 201. The program can also be provided via a network.

### Industrial Applicability

Embodiments of the present invention can be applied to a technique for measuring an RRI.

### Reference Signs List

1 Sensor

2 Signal processing unit

3 Storage unit

4 Communication unit

5 Power supply unit

20 AFE

21 ADC

22 MCU

220 Data storage unit

221 Data acquisition unit

222 Filtering unit

223 Feature point detection unit

224 Time estimation unit

225 RRI calculation unit

226 Heart rate calculation unit.

1.-7. (canceled)

8. An RRI measurement device comprising:
a detection circuit configured to detect a feature point derived from an RS wave from a sampling data sequence of an electrocardiogram signal of a subject;
an estimation circuit configured to:
search for two sampling points from sampling data within a predetermined range of the feature point, wherein a product of adjacent pieces of sampling data to the two sampling points is zero or less; and
estimate a time at which the electrocardiogram signal becomes zero using the two sampling points; and
a calculation circuit configured to calculate an RRI based on time-series data of the time at which the electrocardiogram signal becomes zero as estimated by the estimation circuit.

9. The RRI measurement device according to claim 8, wherein:
the detection circuit is configured to detect a section of an R wave from the sampling data sequence of the electrocardiogram signal and detect any one representative point of pieces of sampling data in the section as the feature point.

10. The RRI measurement device according to claim 8, wherein the detection circuit is configured to:
calculate a data sequence of time difference values from the sampling data sequence of the electrocardiogram signal;
detect a section from the R wave to an S wave from the data sequence of the time difference values; and
detect any one representative point of the time difference values in the section as the feature point.

11. The RRI measurement device according to claim 8, further comprising:
a filtering circuit provided in a preceding stage of the detection circuit and the estimation circuit, the filtering circuit configured to perform an anti-aliasing filtering process or band-pass filtering process on the sampling data sequence of the electrocardiogram signal,
wherein the detection circuit is configured to detect the feature point using the sampling data sequence processed by the filtering circuit, and
wherein the estimation circuit is configured to estimate the time at which the electrocardiogram signal becomes zero using the sampling data sequence processed by the filtering circuit.

12. An RRI measurement device comprising:
a detection circuit configured to detect a feature point derived from an RS wave from a sampling data sequence of an electrocardiogram signal of a subject;
an estimation circuit configured to:
search for two time difference values from time difference values of sampling data within a predetermined range of the feature point, wherein a product of adjacent time difference values to the two time difference values is zero or less; and
estimate a time at which the electrocardiogram signal becomes zero using the two time difference values; and
a calculation circuit configured to calculate an RRI based on time-series data of the time at which the electrocardiogram signal becomes zero as estimated by the estimation circuit.

13. The RRI measurement device according to claim 12, wherein:
the detection circuit is configured to detect a section of an R wave from the sampling data sequence of the electrocardiogram signal and detect any one representative point of pieces of sampling data in the section as the feature point.

14. The RRI measurement device according to claim 12, wherein the detection circuit is configured to:
calculate a data sequence of time difference values from the sampling data sequence of the electrocardiogram signal;
detect a section from the R wave to an S wave from the data sequence of the time difference values; and
detect any one representative point of the time difference values in the section as the feature point.

15. The RRI measurement device according to claim 12, further comprising:
a filtering circuit provided in a preceding stage of the detection circuit and the estimation circuit, the filtering circuit configured to perform an anti-aliasing filtering process or band-pass filtering process on the sampling data sequence of the electrocardiogram signal,
wherein the detection circuit is configured to detect the feature point using the sampling data sequence processed by the filtering circuit, and
wherein the estimation circuit is configured to estimate the time at which the electrocardiogram signal becomes zero using the sampling data sequence processed by the filtering circuit.

16. An RRI measurement method comprising:
detecting a feature point derived from an RS wave from a sampling data sequence of an electrocardiogram signal of a subject;
searching for two sampling points from sampling data within a predetermined range of the feature point, wherein a product of adjacent pieces of sampling data to the two sampling points is zero or less;
estimating a time at which the electrocardiogram signal becomes zero using the two sampling points; and
calculating an RRI on based of time-series data of the time estimated at which the electrocardiogram signal becomes zero.

17. A non-transitory computer readable storage medium storing an RRI measurement program for causing a computer to execute the method according to claim 16.

18. The RRI measurement method according to claim 16, wherein detecting the feature point comprises:
detecting a section of an R wave from the sampling data sequence of the electrocardiogram signal and detect any one representative point of pieces of sampling data in the section as the feature point.
